(19) United States
(12) Patent Application Publication
(10) Pub. No.: US 2007/0298007 A1
(43) Pub. Date: Dec. 27, 2007

(54) SLOW RELEASE BIOCIDAL THERMOPLASTIC COMPOSITIONS AND ARTICLES

(76) Inventors: H. HARALD LUTZMANN, CLEVELAND HTS., OH (US); JOHN T. INGLIS, VERO BEACH, FL (US)

Correspondence Address: D.A. STAUFFER PATENT SERVICES LLC, 1006 MONTFORD ROAD, CLEVLAND HTS., OH, 44121-2016, US

(21) Appl. No.: 11/766,076

(22) Filed: Jun. 20, 2007

Related U.S. Application Data
(60) Provisional application No. 60/805,374, filed on Jun. 21, 2006.

(51) Int. Cl. A61K 31/74; A01P 15/00; A61K 31/765; B32B 27/00; B32B 27/32; C08G 65/22; C08L 23/06

(52) U.S. Cl. 424/78.31; 424/78.37; 442/123; 524/855; 524/878

(57) ABSTRACT

In one aspect, the invention is directed to a biocidal composition, comprising a mixture of (a) a substantially oxidation-resistant thermoplastic polymer having a melting point of about 60° C. to about 100° C.; and (b) a composition for releasing chlorine dioxide gas upon exposure to moisture. In another aspect, the invention provides a polymeric matrix comprising the biocidal composition and having a moisture vapor transmission capability of about 100 to about 1000 grams per 24 hours per square meter per mil of thickness. The invention further provides a manufactured article comprising the biocidal composition, for example manufactured by extrusion, injection molding or blow molding.

### CROSS-REFERENCE TO RELATED APPLICATIONS

This application claims the benefit of U.S. Provisional Patent Application No. 60/805,374, filed Jun. 21, 2006 by Lutzmann, et al.

### BACKGROUND OF THE INVENTION

The antiseptic, bactericidal, fungicidal and viricidal properties of chlorine dioxide (ClO₂) and methods of generating and releasing this strong oxidizing gaseous chemical have been described in the technical and patent literature. For example, the controlled release of biocidal and/or deodorant amounts of chlorine dioxide gas from the reaction of a metal chlorite and a second material such as a dry solid hydrophilic material that reacts with the metal chlorite in the presence of water vapor, but not in the substantial absence of liquid water or water vapor, is known. Slow and extended release of ClO₂ from an acidified expanded amorphous aluminum silicate impregnated with a chlorite salt, in the presence of moisture, is also known. Examples of available commercial products such as these, that release ClO₂ when exposed to moisture, include such trade names as Scentrex™, Aseptrol™, Perlox™, and the like. However, these products typically have a low packing density such that they are dusting rather than free-flowing. Generally, they are available in sealed packaging such as, but not limited to, pouches and the like that protect them from ambient moisture until exposure to moisture for release of the ClO₂ is desired. However, because of the low bulk density and dusting properties of these materials, they can not readily be packaged by commercially available packaging machinery. Moreover, the explosive nature of ClO₂ in high concentrations and in contact with easily oxidized polymers at high temperatures, limits the use of the above-referenced products in extruded, injection-molded or blow-molded products.

### SUMMARY OF THE INVENTION

It was unexpectedly discovered that controlled release of ClO₂ from biocidal compositions, including but not limited to commercially available compositions such as those described above, can be achieved by incorporation of such compositions into a low melting point, substantially oxidation-resistant thermoplastic polymer matrix having a moisture (e.g., water) vapor transmission of about 100 to about 1000 grams per 24 hours per square meter per mil of thickness. A release of ClO₂ from the polymer matrix is then proportional to the ingress of moisture into the matrix as a function of the prevailing ambient relative humidity. Moreover, the use of low melting point, substantially oxidation-resistant thermoplastic polymers allows the production of extruded, injection molded and blow molded products without danger of explosion associated with ClO₂. Embedding the ClO₂ source into a thermoplastic polymer matrix results in a mixture that can be a relatively non-dusting and free-flowing powder.

Thus, in one aspect, the invention is directed to a biocidal composition, comprising a mixture of (a) a substantially oxidation-resistant thermoplastic polymer having a melting point of about 60° C. to about 100° C.; and (b) a composition for releasing chlorine dioxide gas upon exposure to moisture. In another aspect, the invention provides a polymeric matrix comprising the biocidal composition and having a moisture vapor transmission capability of about 100 to about 1000 grams per 24 hours per square meter per mil of thickness. The invention further provides an extruded, injection molded or blow molded article comprising the biocidal composition.

### DETAILED DESCRIPTION OF THE INVENTION

According to aspects of the invention, a composition that releases ClO₂ in proportion to water ingress (i.e., a ClO₂ source, or biocide) is embedded into a thermoplastic polymer matrix in order to control the biocide release rate. In contrast to known products that combine powdered metal chlorites and acid producing ingredients in a polymeric matrix, the present invention can incorporate, into a suitable polymer matrix, commercially available products that, upon contact with moisture, will release chlorine dioxide.

Any such ClO₂ source can be used in the biocide compositions according to the invention including, but not limited to, commercially available ClO₂ sources, such as Scentrex™, Aseptrol™, and Perlox™, or the like. Often, available ClO₂ sources have a very low packing density that results in dusting and not free-flowing powders. According to aspects of the invention, the embedding of such a powder into a thermoplastic polymer matrix provides a substantial increase in the bulk density, making the combination a relatively non-dusting and free-flowing powder mixture, for greater ease of handling and packaging with commercially available packaging machinery.

Suitable thermoplastic polymers for use in biocide compositions according to the invention are those having a low melting point, especially those having a melting point of less than about 100° C., more especially about 60° C. to about 100° C., such that, when heated, the polymer can incorporate the ClO₂ source without degrading it.

The suitable thermoplastic polymers are also selected to have a substantial resistance to oxidation. In view of the explosive character of ClO₂ in high concentrations and in contact with easily oxidized polymers at high temperatures, it was found that low melting point polymers with good oxidation stability are the most suitable for safely incorporation of chlorine dioxide releasing compositions. A non-limiting example of a suitable polymer is a polycaprolactone, such as those obtained from Dow Chemical as TONE P767 or P787. However, many other such polymers are available and are well known to those of ordinary skill in the polymer arts.

Control of water vapor transmission and biocide release rates from the polymeric matrix, especially for preparation of extruded and shaped articles, can be achieved by the selection of combinations of thermoplastic polymers used for the polymer matrix. As a non-limiting example, slowing of water vapor transmission and biocide release rate can be provided by blending a polyolefin (i.e., a polyethylene, polypropylene and/or polystyrene) with the mixture of a polycaprolactone and biocide. Exemplary of suitable polyolefins include, but are not limited to, a low molecular weight (waxy) polyethylene (such as Polywax 500 from Baker Petrolite Polymers Div. of Baker Hughes), a high molecular weight high density polyethylene (such as Microthene F from Eastman Chemicals), and the like. Other examples of suitable polyolefin compounds are also readily available and known. The relative amounts of the polymers can be varied according to the properties of each in order to achieve the desired rate of moisture transmission without undue experimentation.

The biocide compositions according to aspects of the invention can be employed as free-flowing powders or can be formed into molded or extruded articles, films, sheets and the like. Because of the substantial increase in bulk density provided to the biocide by the polymer, the compositions can be packaged by commercially available packaging machinery. Packaging protects and seals the product from moisture, especially ambient relative humidity, until release of the ClO₂ is desired.

In some aspects, the powder form of the invention compositions may be used, for example, in a pouch such as that described in U.S. Pat. No. 6,554,887. In another aspect, the powder can be safely extruded at temperatures below 100° C. into a film that can be used as it is for sterile packaging. In yet another aspect, one side of the biocide film can be adhered, such as by lamination or adhesive, to a second film having a low moisture vapor transmission, and the other side of the biocide film can be adhesively mounted to a non-woven or woven fabric. As a non-limiting example, such a biocide film can comprise a surface of a hospital gown or bed sheeting for use in hospital beds. The biocide compositions in powder form can also be formed into various other articles to be used as biocides, by injection molding, blow molding, or extrusion into a sheet for subsequent thermoforming. However, it is important that all processes of making or using the biocidal compositions and of forming articles from them be conducted at less than 35% relative humidity, preferably at less than 25% relative humidity, to avoid egress of ClO₂.

### EXAMPLES

The following examples illustrate methods of preparation of representative invention biocide compositions containing commercially available ClO₂ sources that are embedded in thermoplastic polymer matrices. The examples illustrate the preparation of free-flowing powders non-dusting powders and the release of ClO₂ therefrom upon exposure to moisture. The examples also illustrate the preparation of film from the powders and the subsequent release of the biocide from the film. The examples further illustrate the use of polyethylene waxes to lower moisture vapor transmission and slow the release rate of the biocide from both powders and films. However, the examples are not intended to be limiting, as other ClO₂ sources, other thermoplastic polymers, other polyolefin polymers, other waxes and other materials suitable to control the rates of moisture vapor transmission and the release rate of the biocides can be employed. The methods are exemplary only and other methods for preparing the biocide compositions of the invention can be determined by those skilled in the art without departing from the scope of the invention herein disclosed and claimed.

### Example 1

To 100 grams of polycaprolactone (TONE P767) were added amounts of a commercial ClO₂ source (Perlox™) to result in Perlox™ concentrations of 10%, 20%, 30% and 40% by weight. The ingredients were both in the form of a fine powder. The ingredients were mixed manually and heated in a forced air oven to 60° C. The mixture was then transferred to an industrial Waring high intensity mixer and mixed at low speed for one minute and at high speed for two minutes. The Perlox™, based on an expanded amorphous aluminum silicate, has an extremely low packing density and absorbed the melting polymer quite rapidly, thus increasing the combination's packing density substantially and creating a free-flowing non-dusting powder.

### Example 2

Five grams of the 10% Perlox™-containing powder from Example 1 were placed into a 2 liter cylindrical polyvinylchloride container together with a moist cotton ball. The container was closed with a screw lid. After standing for 2 hours at room temperature, the container was opened and a strong chlorine dioxide odor was noticed.

### Example 3

Five grams of the 10% Perlox™-containing powder from Example 1 were placed between platens that were heated to 75° C. and pressed at 750 psi. A transparent film of approximately four inches in diameter and approximately seven mils thick was formed. As described in Example 2, the film was placed in a 2 liter cylinder along with a moist cotton ball. After 24 hours a strong odor of chlorine dioxide was noticed upon opening of the container.

### Example 4

To 5 grams of the 10% Perlox™-containing powder from Example 1 were added 3 grams of Polywax 500, a low molecular weight polyethylene having a low melting point (Baker Petrolite Polymers Division of Baker Hughes). This mixture was heated to 60° C. and then mixed for one minute at low speed and two minutes at high speed in the Waring blender. The addition of the polyethylene wax lowered the moisture vapor transmission of the polymer composition and thus served as a means to control the ingress of moisture and consequently the release rate of chlorine dioxide.

### Example 5

To 3 grams of the 40% Perlox™-containing powder from Example 1 were added 6 grams of Microthene F (high molecular weight high density polyethylene). After intensive mixing of the powder blend for one minute each at low and high speed in a Waring blender, an aliquot of 2 grams of the mixture was placed between heated platens of a Burton Press at 260° F. for 5 minutes and at a pressure of 150 psi. A transparent flexible film, measuring 4×4 inches at a thickness of 5 mils was obtained. The low moisture permeability of the polyethylene caused extremely slow release of ClO₂ when the film of this example was exposed to moisture.

### Example 6

To lower the cost of the invention composition, a low molecular weight polyethylene with a low melting point can be substituted for the polycaprolactone, although this also slows the release of biocide from the final product. For example, a 40% concentration by weight of Perlox™ was obtained by combining 4 parts of Perlox™ with 6 parts of Polywax 500. The ingredients were mixed manually and heated in a forced air oven to 60° C. They were then transferred to an industrial Waring high intensity mixer and mixed at low speed for one minute and at high speed for two minutes.

### Example 7

The biocide release rate was slowed even further by combining 3 parts of the material from Example 6 with 6 parts of Microthene F polyethylene.

### Example 8

To 23.25 grams of Perlox were added 5.85 g of polycaprolactone (P767 TONE). The ingredients were mixed in a one liter Waring blender for one minute at low speed and 2 minutes at high speed. The resulting powder was heated at 70° C. for 3 hours to attain a homogenous blend.

The original Perlox™ had a bulk density of 0.093 grams/cc. After mixing with the polycaprolactone, the bulk density of the mixture was increased to 0.44 grams/cc.

While the invention has been described herein with reference to the preferred embodiments, it is to be understood that it is not intended to limit the invention to the specific forms disclosed. On the contrary, it is intended that the invention cover all modifications and alternative forms falling within the scope of the appended claims.

We claim:

1. A biocidal composition, comprising a mixture of
(a) a substantially oxidation-resistant thermoplastic polymer having a melting point of about 60° C. to about 100° C.; and
(b) a composition for releasing chlorine dioxide gas upon exposure to moisture.

2. The composition of claim 1, wherein the thermoplastic polymer is selected from the group consisting of a polycaprolactone, a polyolefin, and mixtures thereof.

3. The composition of claim 1, wherein the composition comprises a physical form selected from the group consisting of a free-flowing powder, an extruded article, an injection molded article, a blow molded article, a film, a sheet, and combinations thereof.

4. A polymeric matrix comprising:
(a) a substantially oxidation-resistant thermoplastic polymer having a melting point of about 60° C. to about 100° C.; and
(b) a composition for releasing chlorine dioxide gas upon exposure to moisture,
wherein the polymeric matrix comprises a moisture vapor transmission capability of about 100 to about 1000 grams per 24 hours per square meter per mil of thickness.

5. The polymeric matrix of claim 4, wherein the thermoplastic polymer is selected from the group consisting of a polycaprolactone, a polyolefin, and mixtures thereof.

6. A manufactured article comprising:
a biocidal composition, comprising a mixture of (a) a substantially oxidation-resistant thermoplastic polymer having a melting point of about 60° C. to about 100° C., and (b) a composition for releasing chlorine dioxide gas upon exposure to moisture,
wherein the article comprises a moisture vapor transmission capability of about 100 to about 1000 grams per 24 hours per square meter per mil of thickness.

7. The article of claim 6, wherein:
the article is manufactured using an extrusion, injection molding, or blow molding process.

8. The article of claim 6, wherein the article comprises a film.

9. The article of claim 8, wherein the article comprises a woven or non-woven fabric comprising said film adhered thereto.
